(19) United States
(12) Patent Application Publication
(10) Pub. No.: US 2018/0256570 A1
(43) Pub. Date: Sep. 13, 2018

(54) TOPICAL FORMULATIONS

(71) Applicant: IGNYTA, INC., San Diego, CA (US)

(72) Inventors: Jeffrey Alan PETERSON, Indianapolis, IN (US); Stephanie Ann SWEETANA, Indianapolis, IN (US)

(73) Assignee: IGNYTA, INC., San Diego, CA (US)

(21) Appl. No.: 15/779,480

(22) PCT Filed: Nov. 28, 2016
(86) PCT No.: PCT/US16/63885

§ 371 Date: May 25, 2018

Related U.S. Application Data
(60) Provisional application No. 62/261,207, filed on Nov. 30, 2015.

(51) Int. Cl. A61K 31/502; A61K 47/38; A61K 47/20; A61K 47/10; A61K 9/00; A61P 35/00

(52) U.S. Cl. CPC A61K 31/502; A61K 47/38; A61P 35/00; A61K 47/10; A61K 9/0014; A61K 47/20

(57) ABSTRACT

Described herein are pharmaceutical formulations comprising 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, wherein the pharmaceutical formulations are suitable for topical use, and methods of using such formulations in the treatment of subjects having cancer.

### CROSS-REFERENCE TO RELATED APPLICATIONS

This application claims the benefit of priority to U.S. Provisional Application No. 62/261,207, filed Nov. 30, 2015, which is hereby incorporated by reference in its entirety.

### BACKGROUND

The present disclosure relates embodiments comprising topical formulations for the treatment of cancer.

### SUMMARY OF THE INVENTION

In one aspect, disclosed herein are topical formulations comprising 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof; one or more diluents; and one or more gelling agents. In some embodiments, 4-fluoro-N-methyl-N-(1-(4-(1-methyl-1H-pyrazol-5-yl)phthalazin-1-yl)piperidin-4-yl)-2-(trifluoromethyl)benzamide is present in an amount of from about 0.1% w/w to about 5% w/w. In some embodiments, 4-fluoro-N-methyl-N-(1-(4-(1-methyl-1H-pyrazol-5-yl)phthalazin-1-yl)piperidin-4-yl)-2-(trifluoromethyl)benzamide is present in an amount of from about 0.4% w/w to about 2% w/w. In some embodiment, the one or more diluents comprise isopropyl alcohol, dimethyl sulfoxide, or a combination thereof. In some embodiments, the one or more diluents are present in an amount of from about 40% w/w to about 99% w/w. In some embodiments, the one or more gelling agents comprise hydroxypropyl cellulose. In some embodiments, the one or more gelling agents are present in an amount of from about 0.5% w/w to about 10% w/w. In some embodiments, the one or more gelling agents are present in an amount of from about 1% w/w to about 3% w/w.

In another aspect, disclosed herein are pharmaceutical formulations comprising 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, and one or more gelling agents, wherein the pharmaceutical formulations are suitable for topical use in a subject. In some embodiments, 4-fluoro-N-methyl-N-(1-(4-(1-methyl-1H-pyrazol-5-yl)phthalazin-1-yl)piperidin-4-yl)-2-(trifluoromethyl)benzamide is present in an amount of from about 0.1% w/w to about 5% w/w. In some embodiments, 4-fluoro-N-methyl-N-(1-(4-(1-methyl-1H-pyrazol-5-yl)phthalazin-1-yl)piperidin-4-yl)-2-(trifluoromethyl)benzamide is present in an amount of from about 0.4% w/w to about 2% w/w. In some embodiments, the one or more gelling agents comprise hydroxypropyl cellulose. In some embodiments, the one or more gelling agents are present in an amount of from about 0.5% w/w to about 10% w/w. In some embodiments, the one or more gelling agents are present in an amount of from about 1% w/w to about 3% w/w. In some embodiments, the pharmaceutical formulation further comprises one or more diluents. In some embodiments, the one or more diluents comprise isopropyl alcohol, dimethyl sulfoxide, or a combination thereof. In some embodiments, the one or more diluents are present in an amount of from about 40% w/w to about 99% w/w.

In another aspect, disclosed herein are methods of treating cancer in a subject, comprising topically administering to the subject a composition comprising a therapeutically effective amount of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, and one or more gelling agents. In some embodiments, the cancer is basal cell carcinoma. In some embodiments, the basal cell carcinoma is selected from nodular, superficial, morpheaform, pigmented basal cell carcinoma, and Fibroepithelioma of Pinkus (FEP). In some embodiments, the composition is administered to the subject once per day. In some embodiments, the composition is administered to the subject once per day for 2 consecutive days. In some embodiments, the composition is administered to the subject following surgery to treat the cancer.

### BRIEF DESCRIPTION OF THE DRAWINGS

FIG. 1 shows the extent of solvolysis with an increase in water concentration in ethanol-based formulations of 4-fluoro-N-methyl-N-(1-(4-(1-methyl-1H-pyrazol-5-yl)phthalazin-1-yl)piperidin-4-yl)-2-(trifluoromethyl)benzamide.

FIG. 2 shows a comparison of compound flux using two different formulations of 4-fluoro-N-methyl-N-(1-(4-(1-methyl-1H-pyrazol-5-yl)phthalazin-1-yl)piperidin-4-yl)-2-(trifluoromethyl)benzamide and a formulation of LDE225.

FIG. 3 shows compound accumulation in epidermis and dermis for two different formulations of 4-fluoro-N-methyl-N-(1-(4-(1-methyl-1H-pyrazol-5-yl)phthalazin-1-yl)piperidin-4-yl)-2-(trifluoromethyl)benzamide compared to a formulation of LDE225.

FIG. 4 shows percent inhibition of Gli1 gene expression from analysis of skin samples from minipigs after administration of one of three different formulations of 4-fluoro-N-methyl-N-(1-(4-(1-methyl-1H-pyrazol-5-yl)phthalazin-1-yl)piperidin-4-yl)-2-(trifluoromethyl)benzamide or a formulation of LDE225.

### DETAILED DESCRIPTION

The Hedgehog (Hh) signaling pathway plays an important role in embryonic pattern formation and adult tissue maintenance by directing cell differentiation and proliferation. The Hedgehog (Hh) protein family, which includes Sonic Hedgehog (Shh), Indian Hedgehog (Ihh), and Desert Hedgehog (Dhh) are secreted glycoproteins that undergo post-translational modifications, including autocatalytic cleavage and coupling of cholesterol to the amino-terminal peptide to form the fragment that possesses signaling activity. Hh binds to the twelve-pass transmembrane protein Ptch (Ptch1 and Ptch2), thereby alleviating Ptch-mediated suppression of Smoothened (Smo). Smo activation triggers a series of intracellular events culminating in the stabilization of the Gli transcription factors (Gli1, Gli2, and Gli3) and the expression of Gli-dependent genes that are responsible for cell proliferation, cell survival, angiogenesis and invasion.

Hh signaling has recently attracted considerable interest based on the discovery that aberrant activation of Shh signaling leads to the formation of various tumors, e.g., pancreatic cancer, medulloblastoma, basal cell carcinoma, small cell lung cancer, and prostate cancer. WO2005033288 discloses certain 1,4-disubstituted phthalazine compounds asserted to be hedgehog antagonists. Similarly, WO2008110611 discloses certain 1,4-disubstituted phthalazine compounds related to the diagnosis and treatment of pathologies related to the hedgehog pathway. WO2009002469 discloses certain 1,4-disubstituted phthalazine compounds that are asserted to be a treatment option for all tumors driven by inappropriate hedgehog signaling.

Provided herein, in one aspect, are pharmaceutical formulations comprising a hedgehog pathway inhibitor (4-fluoro-N-methyl-N-(1-(4-(1-methyl-1H-pyrazol-5-yl)phthalazin-1-yl)piperidin-4-yl)-2-(trifluoromethyl)benzamide) for topical administration to a subject and methods of their preparation and use. In some embodiments, the pharmaceutical formulations leave no scarring, pain, or discomfort for the subject during treatment.

The term “4-fluoro-N-methyl-N-(1-(4-(1-methyl-1H-pyrazol-5-yl)phthalazin-1-yl)piperidin-4-yl)-2-(trifluoromethyl)benzamide” means a compound having the chemical structure:

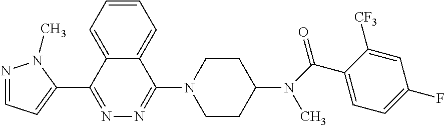 [SMILES: CN(C(=O)c1ccc(F)cc1C(F)(F)F)C1CCN(c2nnc(-c3ccnn3C)c3ccccc23)CC1]

and which is also called taladegib, and has the Chemical Abstracts registry number 1258861-20-9.

As used herein, the terms “cancer” or “tumor” may be used interchangeably. These terms mean the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers. In certain embodiments, the cancer is a solid tumor, a soft tissue tumor, or a metastatic lesion.

As used herein, the term “chemotherapeutic agent”, means a chemical substance, such as a cytotoxic or cytostatic agent, that is used to treat a condition, particularly cancer. In some embodiments, the chemotherapeutic agents include a compound disclosed herein, or a pharmaceutically acceptable salt thereof.

As used herein, the terms “combination” and “in combination with” mean the administration of a first compound together with an at least one additional pharmaceutical or medicinal agent (e.g., an anti-cancer agent), either sequentially or simultaneously. It includes dosing simultaneously, or within minutes or hours of each other, or on the same day, or on alternating days, or dosing a first compound on a daily basis, or multiple days per week, or weekly basis, for example, while administering another compound such as a chemotherapeutic agent on the same day or alternating days or weeks or on a periodic basis during a time simultaneous therewith or concurrent therewith, or at least a part of the time during which the first compound dosed.

As used herein, the term “pharmaceutically acceptable salt” means those salts that retain the biological effectiveness and properties of the parent compound.

The terms “subject” or “patient” as used herein are used interchangeably and mean a mammal, such as a cat, dog, rodent or primate. Typically the subject is a human, and, preferably, a human suffering from or suspected of suffering from the disease and/or disorder.

The term “therapeutically effective amount” as used herein means that amount of the compound or compounds being administered which will relieve to some extent one or more of the symptoms of the disorder being treated. In reference to the treatment of a cancer, a therapeutically effective amount means that amount which has the effect of (1) reducing the size of a cancer tumor, (2) inhibiting (that is, slowing to some extent, preferably stopping) cancer tumor metastasis, (3) inhibiting to some extent (that is, slowing to some extent, preferably stopping) cancer tumor growth, and/or, (4) relieving to some extent (or, preferably, eliminating) one or more symptoms associated with the cancer.

The term “topical administration” means local and/or external application of a pharmaceutical formulation to a subject's body, such as application to the subject's skin.

The term “topical formulation” means a pharmaceutical formulation designed for or involving topical administration to a subject.

The terms “treatment” or “treating” as used herein mean any treatment of a pathologic condition in a subject, and includes: (i) preventing the pathologic condition from occurring in a subject who may be predisposed to the condition but has not yet been diagnosed with the condition and, accordingly, the treatment constitutes as prophylactic treatment for the condition; (ii) inhibiting the pathologic condition, i.e., arresting the development; (iii) relieving the pathologic condition, i.e., causing a regression of the pathologic condition; or (iv) relieving conditions mediated by the pathologic condition.

The preparation of 4-fluoro-N-methyl-N-(1-(4-(1-methyl-1H-pyrazol-5-yl)phthalazin-1-yl)piperidin-4-yl)-2-(trifluoromethyl)benzamide is described in U.S. Pat. No. 8,273,742 and U.S. Pat. No. 9,000,023, both of which are incorporated by reference herein in their entirety.

Unless indicated otherwise, all references herein to 4-fluoro-N-methyl-N-(1-(4-(1-methyl-1H-pyrazol-5-yl)phthalazin-1-yl)piperidin-4-yl)-2-(trifluoromethyl)benzamide include references to salts, solvates, hydrates and complexes thereof, and to solvates, hydrates and complexes of salts thereof, including polymorphs, stereoisomers, and isotopically labeled versions thereof.

4-Fluoro-N-methyl-N-(1-(4-(1-methyl-1H-pyrazol-5-yl)phthalazin-1-yl)piperidin-4-yl)-2-(trifluoromethyl)benzamide may exist in the form of pharmaceutically acceptable salts such as, e.g., acid addition salts of 4-fluoro-N-methyl-N-(1-(4-(1-methyl-1H-pyrazol-5-yl)phthalazin-1-yl)piperidin-4-yl)-2-(trifluoromethyl)benzamide. As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of basic groups which are present in 4-fluoro-N-methyl-N-(1-(4-(1-methyl-1H-pyrazol-5-yl)phthalazin-1-yl)piperidin-4-yl)-2-(trifluoromethyl)benzamide.

For example, 4-fluoro-N-methyl-N-(1-(4-(1-methyl-1H-pyrazol-5-yl)phthalazin-1-yl)piperidin-4-yl)-2-(trifluoromethyl)benzamide is capable of forming a wide variety of salts with various inorganic and organic acids. Although such salts must be pharmaceutically acceptable for administration to a subject (e.g., a mammal), it is often desirable in practice to initially isolate 4-fluoro-N-methyl-N-(1-(4-(1-methyl-1H-pyrazol-5-yl)phthalazin-1-yl)piperidin-4-yl)-2-(trifluoromethyl)benzamide from the reaction mixture as a pharmaceutically unacceptable salt and convert the latter to a free base and subsequently to a pharmaceutically acceptable acid addition salt. The acid addition salts of 4-fluoro-N-methyl-N-(1-(4-(1-methyl-1H-pyrazol-5-yl)phthalazin-1-yl)piperidin-4-yl)-2-(trifluoromethyl)benzamide herein can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

The acids that may be used to prepare pharmaceutically acceptable acid addition salts of such basic compounds of those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts.

Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfate, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, .gamma.-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phosphate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

4-Fluoro-N-methyl-N-(1-(4-(1-methyl-1H-pyrazol-5-yl)phthalazin-1-yl)piperidin-4-yl)-2-(trifluoromethyl)benzamide includes a basic moiety, such as an amino group, that may form pharmaceutically acceptable salts with various amino acids, in addition to the acids mentioned above.

Hemisalts of bases may also be formed, for example, hemisulfate salts.

For a review on suitable salts, see “Handbook of Pharmaceutical Salts: Properties, Selection, and Use” by Stahl and Wermuth (Wiley-VCH, Weinheim, Germany, 2002).

Salts of 4-fluoro-N-methyl-N-(1-(4-(1-methyl-1H-pyrazol-5-yl)phthalazin-1-yl)piperidin-4-yl)-2-(trifluoromethyl)benzamide can be prepared according to methods known to those of skill in the art. A pharmaceutically acceptable salt of 4-fluoro-N-methyl-N-(1-(4-(1-methyl-1H-pyrazol-5-yl)phthalazin-1-yl)piperidin-4-yl)-2-(trifluoromethyl)benzamide can be readily prepared by mixing together solutions of the 4-fluoro-N-methyl-N-(1-(4-(1-methyl-1H-pyrazol-5-yl)phthalazin-1-yl)piperidin-4-yl)-2-(trifluoromethyl)benzamide and the desired acid, as appropriate. The salt may precipitate from solution and be collected by filtration or may be recovered by evaporation of the solvent. The degree of ionization in the salt may vary from completely ionized to almost non-ionized.

It will be understood by those of skill in the art that 4-fluoro-N-methyl-N-(1-(4-(1-methyl-1H-pyrazol-5-yl)phthalazin-1-yl)piperidin-4-yl)-2-(trifluoromethyl)benzamide in free base form having a basic functionality may be converted to the acid addition salts by treating with a stoichiometric excess of the appropriate acid. The acid addition salts of 4-fluoro-N-methyl-N-(1-(4-(1-methyl-1H-pyrazol-5-yl)phthalazin-1-yl)piperidin-4-yl)-2-(trifluoromethyl)benzamide may be reconverted to the corresponding free base by treating with a stoichiometric excess of a suitable base, such as potassium carbonate or sodium hydroxide, typically in the presence of aqueous solvent, and at a temperature of between about 0° C. and 100° C. The free base form may be isolated by conventional means, such as extraction with an organic solvent. In addition, acid addition salts of 4-fluoro-N-methyl-N-(1-(4-(1-methyl-1H-pyrazol-5-yl)phthalazin-1-yl)piperidin-4-yl)-2-(trifluoromethyl)benzamide may be interchanged by taking advantage of differential solubilities of the salts, volatilities or acidities of the acids, or by treating with the appropriately loaded ion exchange resin. For example, the interchange may be affected by the reaction of a salt of 4-fluoro-N-methyl-N-(1-(4-(1-methyl-1H-pyrazol-5-yl)phthalazin-1-yl)piperidin-4-yl)-2-(trifluoromethyl)benzamide with a slight stoichiometric excess of an acid of a lower pK than the acid component of the starting salt. This conversion is typically carried out at a temperature between about 0° C. and the boiling point of the solvent being used as the medium for the procedure.

Pharmaceutically acceptable salts of 4-fluoro-N-methyl-N-(1-(4-(1-methyl-1H-pyrazol-5-yl)phthalazin-1-yl)piperidin-4-yl)-2-(trifluoromethyl)benzamide may be prepared by one or more of the following methods: (i) by reacting 4-fluoro-N-methyl-N-(1-(4-(1-methyl-1H-pyrazol-5-yl)phthalazin-1-yl)piperidin-4-yl)-2-(trifluoromethyl)benzamide with the desired acid; (ii) by removing an acid- or base-labile protecting group from a suitable precursor of 4-fluoro-N-methyl-N-(1-(4-(1-methyl-1H-pyrazol-5-yl)phthalazin-1-yl)piperidin-4-yl)-2-(trifluoromethyl)benzamide or by ring-opening a suitable cyclic precursor, for example, a lactone or lactam, using the desired acid or base; or (iii) by converting one salt of 4-fluoro-N-methyl-N-(1-(4-(1-methyl-1H-pyrazol-5-yl)phthalazin-1-yl)piperidin-4-yl)-2-(trifluoromethyl)benzamide to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionized to almost non-ionized.

The compounds disclosed herein may exist in both unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm. The term “solvate” is used herein to describe a molecular complex comprising 4-fluoro-N-methyl-N-(1-(4-(1-methyl-1H-pyrazol-5-yl)phthalazin-1-yl)piperidin-4-yl)-2-(trifluoromethyl)benzamide, or a salt thereof, and one or more pharmaceutically acceptable solvent molecules, for example, ethanol. The term “hydrate” is employed when the solvent is water. Pharmaceutically acceptable solvates in accordance with the embodiments disclosed herein include hydrates and solvates wherein the solvent of crystallization may be isotopically substituted, e.g., D₂O, d₆-acetone, d₆-DMSO.

Also included within the scope disclosed herein are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see Haleblian, J. Pharm. Sci., 1975, 64 (8):1269-1288, the disclosure of which is incorporated herein by reference in its entirety.

Hereinafter all references to compounds disclosed herein include references to salts, solvates and complexes thereof and to solvates and complexes of salts thereof.

The compounds disclosed herein include all polymorphs and crystal habits thereof, prodrugs and isomers thereof (including optical, geometric and tautomeric isomers) as hereinafter defined and isotopically-labeled compounds disclosed herein.

4-Fluoro-N-methyl-N-(1-(4-(1-methyl-1H-pyrazol-5-yl)phthalazin-1-yl)piperidin-4-yl)-2-(trifluoromethyl)benzamide may exhibit the phenomena of tautomerism and structural isomerism. For example, the compounds may exist in several tautomeric forms, including the enol and imine form, and the keto and enamine form and geometric isomers and mixtures thereof. All such tautomeric forms are included within the scope of compounds disclosed herein. Tautomers exist as mixtures of a tautomeric set in solution. In solid form, usually one tautomer predominates. Even though one tautomer may be described, the compounds disclosed herein are meant to encompass all tautomers of these compounds.

4-Fluoro-N-methyl-N-(1-(4-(1-methyl-1H-pyrazol-5-yl)phthalazin-1-yl)piperidin-4-yl)-2-(trifluoromethyl)benzamide intended for pharmaceutical use may be administered as crystalline or amorphous products, or mixtures thereof. They may be obtained, for example, as solid plugs, powders, or films by methods such as precipitation, crystallization, freeze drying, spray drying, or evaporative drying. Microwave or radio frequency drying may be used for this purpose.

In some embodiments, 4-fluoro-N-methyl-N-(1-(4-(1-methyl-1H-pyrazol-5-yl)phthalazin-1-yl)piperidin-4-yl)-2-(trifluoromethyl)benzamide is present in the composition or formulation in an amount of from about 0.1% w/w to about 5% w/w. In some embodiments, 4-fluoro-N-methyl-N-(1-(4-(1-methyl-1H-pyrazol-5-yl)phthalazin-1-yl)piperidin-4-yl)-2-(trifluoromethyl)benzamide is present in the composition or formulation in an amount of from about 0.4% w/w to about 2% w/w. In some embodiments, 4-fluoro-N-methyl-N-(1-(4-(1-methyl-1H-pyrazol-5-yl)phthalazin-1-yl)piperidin-4-yl)-2-(trifluoromethyl)benzamide is present in the composition or formulation at about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% w/w, or more, including increments thereof.

In some embodiments, 4-fluoro-N-methyl-N-(1-(4-(1-methyl-1H-pyrazol-5-yl)phthalazin-1-yl)piperidin-4-yl)-2-(trifluoromethyl)benzamide is topically administered to a subject in a composition or formulation comprising one or more gelling agents.

Some embodiments relate to the use of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, in the manufacture of a medicament for the treatment of cancer in a subject.

Some embodiments relate to 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, for use as a medicament.

Some embodiments relate to pharmaceutical formulations comprising 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof (e.g., pharmaceutical formulations). In some embodiments, the pharmaceutical formulation comprises 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, and one or more gelling agents. In some embodiments, the pharmaceutical formulation comprises 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, one or more gelling agents, and one or more diluents. In another embodiment are provided pharmaceutical formulations comprising 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt, one or more pharmaceutically acceptable carriers and, optionally, at least one additional medicinal or pharmaceutical agent. In some embodiments, the at least one additional medicinal or pharmaceutical agent is an anti-cancer agent selected from 5-fluorouracil, vismodegib, sonidegib, and imiquimod. In some embodiments, the one additional medicinal or pharmaceutical agent is 5-fluorouracil. In some embodiments, the one additional medicinal or pharmaceutical agent is vismodegib. In some embodiments, the one additional medicinal or pharmaceutical agent is sonidegib. In some embodiments, the one additional medicinal or pharmaceutical agent is imiquimod.

Some embodiments relate to pharmaceutical formulations consisting essentially of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof (e.g., pharmaceutical formulations). In some embodiments, the pharmaceutical formulation consists essentially of -fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, and one or more gelling agents. In some embodiments, the pharmaceutical formulation consists essentially of -fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, one or more gelling agents, and one or more diluents. In another embodiment are provided pharmaceutical formulations consisting essentially 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt, one or more pharmaceutically acceptable carriers and, optionally, at least one additional medicinal or pharmaceutical agent. In some embodiments, the at least one additional medicinal or pharmaceutical agent is an anti-cancer agent selected from 5-fluorouracil, vismodegib, sonidegib, and imiquimod. In some embodiments, the one additional medicinal or pharmaceutical agent is 5-fluorouracil. In some embodiments, the one additional medicinal or pharmaceutical agent is vismodegib. In some embodiments, the one additional medicinal or pharmaceutical agent is sonidegib. In some embodiments, the one additional medicinal or pharmaceutical agent is imiquimod.

In some embodiments, the pharmaceutically acceptable carrier comprises a conventional pharmaceutical carrier or excipient. Suitable pharmaceutical carriers include inert diluents or fillers, water and various organic solvents (such as hydrates and solvates). The pharmaceutical compositions or formulations may, if desired, contain additional ingredients such as gelling agents, binders, excipients and the like.

In some embodiments, 4-fluoro-N-methyl-N-(1-(4-(1-methyl-1H-pyrazol-5-yl)phthalazin-1-yl)piperidin-4-yl)-2-(trifluoromethyl)benzamide is delivered to the subject via local, topical or percutaneous delivery with suitable excipients or carriers to enable and/or enhance drug penetration. Suitable carriers or excipients may enhance the physical and chemical stability of the formulation or enhance its aesthetic properties.

The carrier may be any gel, ointment, lotion, emulsion, cream, foam, mousse, liquid, spray, or aerosol which is capable of delivering the drug to the skin of the subject. In the local drug delivery vehicles described herein, a compounding agent, co-solvent, surfactant, emulsifier, antioxidant, preservative, stabilizer, or diluent may be included in the formulation. A suitable emulsifying agent is needed if the active agent is insoluble in an aqueous environment. A penetration enhancer may be added to enable the active agent to cross the barrier of the stratum corneum. In some embodiments, the carrier is a gel, which is odorless and tasteless and dissolves rapidly, such as a hydroalcoholic gel.

In some embodiments, the pharmaceutical composition or formulation comprises 4-fluoro-N-methyl-N-(1-(4-(1-methyl-1H-pyrazol-5-yl)phthalazin-1-yl)piperidin-4-yl)-2-(trifluoromethyl)benzamide and one or more diluents. In some embodiments, diluents are included in the formulations to dissolve, disperse or otherwise incorporate the carrier. Examples of diluents include, but are not limited to, water, buffered aqueous solutions, organic hydrophilic diluents, such as monovalent alcohols, and low molecular weight glycols and polyols (e.g., propylene glycol, polypropylene glycol, glycerol, butylene glycol). In some embodiments, the one or more diluents comprise isopropyl alcohol, dimethyl sulfoxide (DMSO), or a combination thereof. In some embodiments, the one or more diluents are present in an amount of from about 20% w/w to about 99% w/w. In some embodiments, the one or more diluents are present in an amount of from about 40% w/w to about 99% w/w. In some embodiments, each of the one or more diluents are present in an amount of least at about 5% w/w. In some embodiments, each of the one or more diluents are present in an amount of least at about 10% w/w. In some embodiments, each of the one or more diluents are present in an amount of least at about 20% w/w. In some embodiments, each of the one or more diluents are present in an amount of least at about 30% w/w. In some embodiments, each of the one or more diluents are present in an amount of least at about 40% w/w. In some embodiments, one or more diluents are present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% w/w, including increments thereof.

Appropriate excipients are selected based on the active agent and the type of the formulation. Standard excipients include, but are not limited to, gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethyl cellulose calcium, carboxymethyl cellulose sodium, methylcellulose, hydroxyethyl cellulose, hydroxypropyl cellulose, hydroxypropylmethyl cellulose phthalate, noncrystalline cellulose, magnesium aluminum silicate, triethanolamine, polyvinyl alcohol, polyvinylpyrrolidone, sugars, and starches.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In some embodiments, the composition or formulation further comprises one or more emollients.

In some embodiments, the composition or formulation further comprises one or more buffers. In some embodiments, the one or more buffers maintain the composition or formulation at a pH of from about 4 to about 7.5. In some embodiments, the one or more buffers maintain the composition or formulation at a pH of from about 4 to about 7. In some embodiments, the one or more buffers maintain the composition or formulation at a pH of from about 5 to about 7.

In some embodiments, the composition or formulation further comprises one or more penetration enhancers. Penetration enhancers are frequently used to promote transdermal delivery of drugs across the skin, in particular across the stratum corneum. Some penetration enhancers cause dermal irritation, dermal toxicity and dermal allergies. However, the more commonly used ones include, but are not limited to, dimethyl sulfoxide, urea, (carbonyldiamide), imidurea, N,N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). In some embodiments, the one or more penetration enhancer comprises dimethyl sulfoxide.

In some embodiments, the formulation is a gel. A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Examples of the formulation of 4-fluoro-N-methyl-N-(1-(4-(1-methyl-1H-pyrazol-5-yl)phthalazin-1-yl)piperidin-4-yl)-2-(trifluoromethyl)benzamide gels are shown in the examples. In some embodiments, the composition or formulation comprising 4-fluoro-N-methyl-N-(1-(4-(1-methyl-1H-pyrazol-5-yl)phthalazin-1-yl)piperidin-4-yl)-2-(trifluoromethyl)benzamide further comprises one or more gelling agents. In some embodiments, the one or more gelling agents are natural, semi-synthetic, or synthetic. Suitable thickening or gelling agents include, but are not limited to, acacia, acrylates/steareth-20 methacrylate copolymer, agar, algin, alginic acid, ammonium acrylate copolymers, ammonium alginate, ammonium chloride, ammonium sulfate, amylopectin, attapulgite, bentonite, C₉-C₁₅ alcohols, calcium acetate, calcium alginate, calcium carrageenan, calcium chloride, caprylic alcohol, vinyl polymers such as cross linked acrylic acid polymers with the name carbomer, such as but not limited to carbomer 910, carbomer 934, carbomer 934P, carbomer 940, carbomer 941; modified celluloses such as hydroxypropyl cellulose and hydroxyethyl cellulose; Carbopol homopolymers and copolymers, carboxymethyl hydroxyethylcellulose, carboxymethyl hydroxypropyl guar, carrageenan, cellulose, cellulose gum, cetearyl alcohol, cetyl alcohol, corn starch, damar, dextrin, dibenzylidine sorbitol, ethylene dihydrogenated tallowamide, ethylene dioleamide, ethylene distearamide, gelatin, guar gum, hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, hydroxypropylcellulose, hydroxypropyl guar, hydroxypropyl methylcellulose, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M is also known as Polyox WSR® N-IO, which is available from Union Carbide and as PEG-2,000; PEG-5M is also known as Polyox WSR® N-35 and Polyox WSR® N-80, both available from Union Carbide and as PEG-5,000 and Polyethylene Glycol 300,000; PEG-7M is also known as Polyox WSR® N-750 available from Union Carbide; PEG 9-M is also known as Polyox WSR® N-3333 available from Union Carbide; PEG-14M is also known as Polyox WSR® N-3000 available from Union Carbide, polyacrylic acid, polyvinyl alcohol, potassium alginate, potassium aluminum polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacrylate, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, and mixtures thereof. In some embodiments, the one or more gelling agents comprise hydroxypropyl cellulose (HPC).

The concentration of one or more gelling agents can be adjusted to change the viscosity of the gel. For example, in some embodiments the formulation includes less than 1%, or about 1%, 2%, 3%, 4%, 5%, 6%, 7%, 8%, 9%, 10%, 20%, 30%, 40%, 50%, 60%, 70%, or 80% w/w, including increments therein, of the one or more gelling agents. In some embodiments, the one or more gelling agents are present in an amount of from about 0.1% w/w to about 80% w/w. In some embodiments, the one or more gelling agents are present in an amount of from about 0.5% w/w to about 10% w/w. In some embodiments, the one or more gelling agents are present in an amount of from about 0.5% w/w to about 5% w/w. In some embodiments, the one or more gelling agents are present in an amount of from about 1% w/w to about 3% w/w.

In some embodiments, the composition or formulation has a viscosity of at least 100 cP. In some embodiments, the composition or formulation has a viscosity of at least 500 cP. In some embodiments, the composition or formulation has a viscosity of about 100 cP to about 20,000 cP. In some embodiments, the composition or formulation has a viscosity of about 100 cP to about 15,000 cP. In some embodiments, the composition or formulation has a viscosity of about 100 cP to about 10,000 cP. In some embodiments, the composition or formulation has a viscosity of about 100 cP to about 5,000 cP. In some embodiments, the composition or formulation has a viscosity of about 500 cP to about 5,000 cP. In some embodiments, the composition or formulation has a viscosity of about 100, 200, 300, 400, 500, 600, 700, 800, 900, 1000, 1500, 2000, 2500, 3000, 3500, 4000, 4500, or 5000 cP, or more, including increments therein. In some embodiments, the composition or formulation is a pseudoplastic fluid (i.e., a fluid that can change viscosity depending on temperature, shear rate, and force).

In some embodiments, the composition or formulation further comprises one or more preservatives. Preservatives are used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In some embodiments, the composition or formulation degrades by less than 1% over the course of 6 months at room temperature. More preferably, the rate of degradation is less than 0.9, 0.8, 0.7, 0.6, 0.5, 0.4, 0.3, 0.2, or less than 0.1%, and all fractions in between, over the course of 6 months at room temperature.

In some embodiments, the composition or formulation has a drying time of about four minutes or less. In some embodiments, the composition or formulation has a drying time of about three minutes or less. In some embodiments, the composition or formulation has a drying time of about two and half minutes.

In some embodiments, the composition or formulation delivers a dose of 4-fluoro-N-methyl-N-(1-(4-(1-methyl-1H-pyrazol-5-yl)phthalazin-1-yl)piperidin-4-yl)-2-(trifluoromethyl)benzamide to external and localized areas of the epidermis.

In some embodiments, the compositions or formulations are administered to a subject in need thereof in an amount that contains low dosages of 4-fluoro-N-methyl-N-(1-(4-(1-methyl-1H-pyrazol-5-yl)phthalazin-1-yl)piperidin-4-yl)-2-(trifluoromethyl)benzamide. In some embodiments, the dosage range is from about 0.1 to about 200 mg of 4-fluoro-N-methyl-N-(1-(4-(1-methyl-1H-pyrazol-5-yl)phthalazin-1-yl)piperidin-4-yl)-2-(trifluoromethyl)benzamide. In some embodiments, the dosage range is from about 0.5 to about 100 mg of 4-fluoro-N-methyl-N-(1-(4-(1-methyl-1H-pyrazol-5-yl)phthalazin-1-yl)piperidin-4-yl)-2-(trifluoromethyl)benzamide. In some embodiments, the dosage range is from about 1 to about 50 mg of 4-fluoro-N-methyl-N-(1-(4-(1-methyl-1H-pyrazol-5-yl)phthalazin-1-yl)piperidin-4-yl)-2-(trifluoromethyl)benzamide. In some embodiments, a single dosage is about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 mg, including increments therein, of 4-fluoro-N-methyl-N-(1-(4-(1-methyl-1H-pyrazol-5-yl)phthalazin-1-yl)piperidin-4-yl)-2-(trifluoromethyl)benzamide.

In some embodiments, the composition or formulation is administered once a day to a subject in need thereof. In some embodiments, the composition or formulation is administered twice a day to a subject in need thereof. In some embodiments, the composition or formulation is administered three times a day to a subject in need thereof. In some embodiments, the composition or formulation is administered four times a day to a subject in need thereof. In some embodiments, the composition or formulation is administered five times a day to a subject in need thereof. In some embodiments, the composition or formulation is administered six times a day to a subject in need thereof. In some embodiments, the composition or formulation is administered seven times a day to a subject in need thereof. In some embodiments, the composition or formulation is administered eight times a day to a subject in need thereof. In some embodiments, the composition or formulation is administered nine times a day to a subject in need thereof. In some embodiments, the composition or formulation is administered ten times a day to a subject in need thereof.

In some embodiments, the composition or formulation is administered once a day for 2 consecutive days to a subject in need thereof. In some embodiments, the composition or formulation is administered once a day for 3 consecutive days to a subject in need thereof. In some embodiments, the composition or formulation is administered once a day for 4 consecutive days to a subject in need thereof. In some embodiments, the composition or formulation is administered once a day for 5 consecutive days to a subject in need thereof. In some embodiments, the composition or formulation is administered once a day for 6 consecutive days to a subject in need thereof. In some embodiments, the composition or formulation is administered once a day for 7 consecutive days to a subject in need thereof. In some embodiments, the composition or formulation is administered once a day for 8 consecutive days to a subject in need thereof. In some embodiments, the composition or formulation is administered once a day for 9 consecutive days to a subject in need thereof. In some embodiments, the composition or formulation is administered once a day for 10 consecutive days to a subject in need thereof. In some embodiments, the composition or formulation is administered once a day for 2, 3, 4, 5, 6, 7, 8, 9, 10, 11, 12, 13, 14, 15, 16, 17, 18, 19, 20, 21, 22, 23, 24, 25, 26, 27, 28, 29, or 30 consecutive days, or more, to a subject in need thereof. In some embodiments, the composition or formulation is administered at least once a day for 2, 3, 4, 5, 6, 7, 8, 9, 10, 11, 12, 13, 14, 15, 16, 17, 18, 19, 20, 21, 22, 23, 24, 25, 26, 27, 28, 29, or 30 consecutive days, or more, to a subject in need thereof. In some embodiments, the composition or formulation is administered at least once a day for up to 12 weeks, or more, to a subject in need thereof.

In some embodiments, the composition or formulation is administered to the subject following surgery to treat cancer.

The skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the presently disclosed methods.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions or formulations, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. The embodiments disclosed herein are intended to encompass intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

In another aspect, provided herein are methods for the treatment of cancer in a subject in need thereof, the method comprising topically administering to the subject a composition disclosed herein. In some embodiments, the cancer is skin cancer. In further embodiments, the skin cancer is basal cell carcinoma, melanoma, or squamous cell carcinoma. In some embodiments, the cancer is basal cell carcinoma. In some embodiments, the basal cell carcinoma is selected from nodular, superficial, morpheaform, pigmented basal cell carcinoma, and Fibroepithelioma of Pinkus (FEP). In some embodiments, the basal cell carcinoma is nodular. In some embodiments, the basal cell carcinoma is superficial. In some embodiments, the basal cell carcinoma is morpheaform. In some embodiments, the basal cell carcinoma is pigmented basal cell carcinoma. In some embodiments, the basal cell carcinoma is Fibroepithelioma of Pinkus (FEP). In some embodiments, the composition is administered to the subject following surgery to treat the cancer.

In another aspect, provided herein are methods for the treatment of cancer in a subject in need thereof, the method comprising topically administering to the subject a composition comprising a therapeutically effective amount of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, and one or more gelling agents. In some embodiments, the cancer is skin cancer. In further embodiments, the skin cancer is basal cell carcinoma, melanoma, or squamous cell carcinoma. In some embodiments, the cancer is basal cell carcinoma. In some embodiments, the basal cell carcinoma is selected from nodular, superficial, morpheaform, pigmented basal cell carcinoma, and Fibroepithelioma of Pinkus (FEP). In some embodiments, the basal cell carcinoma is nodular. In some embodiments, the basal cell carcinoma is superficial. In some embodiments, the basal cell carcinoma is morpheaform. In some embodiments, the basal cell carcinoma is pigmented basal cell carcinoma. In some embodiments, the basal cell carcinoma is Fibroepithelioma of Pinkus (FEP). In some embodiments, the composition is administered to the subject following surgery to treat the cancer.

In another aspect, provided herein are methods for the treatment of cancer in a subject in need thereof, the method comprising topically administering to the subject a composition comprising a therapeutically effective amount of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, one or more gelling agents, and one or more diluents. In some embodiments, the cancer is skin cancer. In further embodiments, the skin cancer is basal cell carcinoma, melanoma, or squamous cell carcinoma. In some embodiments, the cancer is basal cell carcinoma. In some embodiments, the basal cell carcinoma is selected from nodular, superficial, morpheaform, pigmented basal cell carcinoma, and Fibroepithelioma of Pinkus (FEP). In some embodiments, the basal cell carcinoma is nodular. In some embodiments, the basal cell carcinoma is superficial. In some embodiments, the basal cell carcinoma is morpheaform. In some embodiments, the basal cell carcinoma is pigmented basal cell carcinoma. In some embodiments, the basal cell carcinoma is Fibroepithelioma of Pinkus (FEP). In some embodiments, the composition is administered to the subject following surgery to treat the cancer.

In another aspect, provided herein are methods for the treatment of cancer in a subject in need thereof, the method comprising topically administering to the subject a composition consisting essentially of a therapeutically effective amount of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, and one or more gelling agents. In some embodiments, the cancer is skin cancer. In further embodiments, the skin cancer is basal cell carcinoma, melanoma, or squamous cell carcinoma. In some embodiments, the cancer is basal cell carcinoma. In some embodiments, the basal cell carcinoma is selected from nodular, superficial, morpheaform, pigmented basal cell carcinoma, and Fibroepithelioma of Pinkus (FEP). In some embodiments, the basal cell carcinoma is nodular. In some embodiments, the basal cell carcinoma is superficial. In some embodiments, the basal cell carcinoma is morpheaform. In some embodiments, the basal cell carcinoma is pigmented basal cell carcinoma. In some embodiments, the basal cell carcinoma is Fibroepithelioma of Pinkus (FEP). In some embodiments, the composition is administered to the subject following surgery to treat the cancer.

In another aspect, provided herein are methods for the treatment of cancer in a subject in need thereof, the method comprising topically administering to the subject a composition consisting essentially of a therapeutically effective amount of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, one or more gelling agents, and one or more diluents. In some embodiments, the cancer is skin cancer. In further embodiments, the skin cancer is basal cell carcinoma, melanoma, or squamous cell carcinoma. In some embodiments, the cancer is basal cell carcinoma. In some embodiments, the basal cell carcinoma is selected from nodular, superficial, morpheaform, pigmented basal cell carcinoma, and Fibroepithelioma of Pinkus (FEP). In some embodiments, the basal cell carcinoma is nodular. In some embodiments, the basal cell carcinoma is superficial. In some embodiments, the basal cell carcinoma is morpheaform. In some embodiments, the basal cell carcinoma is pigmented basal cell carcinoma. In some embodiments, the basal cell carcinoma is Fibroepithelioma of Pinkus (FEP). In some embodiments, the composition is administered to the subject following surgery to treat the cancer.

In another aspect, provided herein are methods for the treatment of basal cell carcinoma in a subject in need thereof, the method comprising topically administering to the subject a composition comprising a therapeutically effective amount of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, and one or more gelling agents. In some embodiments, the basal cell carcinoma is selected from nodular, superficial, morpheaform, pigmented basal cell carcinoma, and Fibroepithelioma of Pinkus (FEP). In some embodiments, the basal cell carcinoma is nodular. In some embodiments, the basal cell carcinoma is superficial. In some embodiments, the basal cell carcinoma is morpheaform. In some embodiments, the basal cell carcinoma is pigmented basal cell carcinoma. In some embodiments, the basal cell carcinoma is Fibroepithelioma of Pinkus (FEP). In some embodiments, the composition is administered to the subject following surgery to treat the cancer.

In another aspect, provided herein are methods for the treatment of basal cell carcinoma in a subject in need thereof, the method comprising topically administering to the subject a composition comprising a therapeutically effective amount of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, one or more gelling agents, and one or more diluents. In some embodiments, the basal cell carcinoma is selected from nodular, superficial, morpheaform, pigmented basal cell carcinoma, and Fibroepithelioma of Pinkus (FEP). In some embodiments, the basal cell carcinoma is nodular. In some embodiments, the basal cell carcinoma is superficial. In some embodiments, the basal cell carcinoma is morpheaform. In some embodiments, the basal cell carcinoma is pigmented basal cell carcinoma. In some embodiments, the basal cell carcinoma is Fibroepithelioma of Pinkus (FEP). In some embodiments, the composition is administered to the subject following surgery to treat the cancer.

In another aspect, provided herein are methods for the treatment of cancer in a subject comprising administering a combination of a topical formulation comprising 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, and at least one additional medicinal or pharmaceutical agent that is administered to said subject other than by topical administration. For example, said at least one additional medicinal or pharmaceutical agent may be administered to said subject orally or intravenously.

In another aspect, provided herein are such methods wherein said at least one additional medicinal or pharmaceutical agent is selected from an anti-angiogenesis agent (e.g., an agent that stops tumors from developing new blood vessels). Examples of anti-angiogenesis agents include for example VEGF inhibitors, VEGFR inhibitors, TIE-2 inhibitors, PDGFR inhibitors, angiopoetin inhibitors, PKC.beta. inhibitors, COX-2 (cyclooxygenase II) inhibitors, integrins (alpha-v/beta-3), MMP-2 (matrix-metalloprotienase 2) inhibitors, and MMP-9 (matrix-metalloprotienase 9) inhibitors. Preferred anti-angiogenesis agents include sunitinib (Sutent®), bevacizumab (Avastin®), axitinib (AG 13736), SU 14813 (Pfizer), and AG 13958 (Pfizer).

Additional anti-angiogenesis agents include vatalanib (CGP 79787), Sorafenib (Nexavar®), pegaptanib octasodium (Macugen®), vandetanib (Zactima®), PF-0337210 (Pfizer), SU 14843 (Pfizer), AZD 2171 (AstraZeneca), ranibizumab (Lucentis®), Neovastat® (AE 941), tetrathiomolybdata (Coprexa®), AMG 706 (Amgen), VEGF Trap (AVE 0005), CEP 7055 (Sanofi-Aventis), XL 880 (Exelixis), telatinib (BAY 57-9352), and CP-868,596 (Pfizer).

Other anti-angiogenesis agents include enzastaurin (LY 317615), midostaurin (CGP 41251), perifosine (KRX 0401), teprenone (Selbex®) and UCN 01 (Kyowa Hakko).

Other examples of anti-angiogenesis agents which can be used as described herein include celecoxib (Celebrex®), parecoxib (Dynastat®), deracoxib (SC 59046), lumiracoxib (Preige®), valdecoxib (Bextra®), rofecoxib (Vioxx®), iguratimod (Careram®), IP 751 (Invedus), SC-58125 (Pharmacia) and etoricoxib (Arcoxia®).

Other anti-angiogenesis agents include exisulind (Aptosyn®), salsalate (Amigesic®), diflunisal (Dolobid®), ibuprofen (Motrin®), ketoprofen (Orudis®) nabumetone (Relafen®), piroxicam (Feldene®), naproxen (Aleve®, Naprosyn®) diclofenac (Voltaren®), indomethacin (Indocin®), sulindac (Clinoril®), tolmetin (Tolectin®), etodolac (Lodine®), ketorolac (Toradol®), and oxaprozin (Daypro®).

Other anti-angiogenesis agents include ABT 510 (Abbott), apratastat (TMI 005), AZD 8955 (AstraZeneca), incyclinide (Metastat®), and PCK 3145 (Procyon).

Other anti-angiogenesis agents include acitretin (Neotigason®), plitidepsin (Aplidine®), cilengtide (EMD 121974), combretastatin A4 (CA4P), fenretinide (4 HPR), halofuginone (Tempostatin®), Panzem® (2-methoxyestradiol), PF-03446962 (Pfizer), rebimastat (BMS 275291), catumaxomab (Removab®), lenalidomide (Revlimid®) squalamine (EVIZON®), thalidomide (Thalomid®), Ukrain® (NSC 631570), Vitaxin® (MEDI 522), and zoledronic acid (Zometa®).

In some embodiments, the at least one additional medicinal or therapeutic agent is a so-called signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, ALK inhibitors, ROS1 inhibitors, TrkA inhibitors, TrkB inhibitors, TrkC inhibitors, farnesyl protein transferase inhibitors, EGF inhibitor, ErbB-1 (EGFR), ErbB-2, pan erb, IGF1R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WNT pathway and so called multi-targeted kinase inhibitors.

Preferred signal transduction inhibitors include gefitinib (Iressa®), cetuximab (Erbitux®), erlotinib (Tarceva®), trastuzumab (Herceptin®), sunitinib (Sutent®) imatinib (Gleevec®), and PD325901 (Pfizer).

Additional examples of signal transduction inhibitors which may be used according to the methods described herein include BMS 214662 (Bristol-Myers Squibb), lonafarnib (Sarasar®), pelitrexol (AG 2037), matuzumab (EMD 7200), nimotuzumab (TheraCIM h-R3®), panitumumab (Vectibix®), Vandetanib (Zactima®), pazopanib (SB 786034), ALT 110 (Alteris Therapeutics), BIBW 2992 (Boehringer Ingelheim), and Cervene® (TP 38).

Other examples of signal transduction inhibitor include PF-2341066 (Pfizer), PF-299804 (Pfizer), canertinib (CI 1033), pertuzumab (Omnitarg®), Lapatinib (Tycerb®), pelitinib (EKB 569), miltefosine (Miltefosin®), BMS 599626 (Bristol-Myers Squibb), Lapuleucel-T (Neuvenge®), NeuVax® (E75 cancer vaccine), Osidem® (IDM 1), mubritinib (TAK-165), CP-724,714 (Pfizer), panitumumab (Vectibix®), lapatinib (Tycerb®), PF-299804 (Pfizer), pelitinib (EKB 569), and pertuzumab (Omnitarg®).

Other examples of signal transduction inhibitors include ARRY 142886 (Array Biopharm), everolimus (Certican®), zotarolimus (Endeavor®), temsirolimus (Torisel®), AP 23573 (ARIAD), and VX 680 (Vertex).

Additionally, other signal transduction inhibitors include XL 647 (Exelixis), sorafenib (Nexavar®), LE-AON (Georgetown University), and GI-4000 (GlobeImmune).

Other signal transduction inhibitors include ABT 751 (Abbott), alvocidib (flavopiridol), BMS 387032 (Bristol Myers), EM 1421 (Erimos), indisulam (E 7070), seliciclib (CYC 200), BIO 112 (One Bio), BMS 387032 (Bristol-Myers Squibb), PD 0332991 (Pfizer), AG 024322 (Pfizer), LOXO-101 (Loxo Oncology), crizotinib, and ceritinib.

In some embodiments, the at least one additional medicinal or therapeutic agent is a classical antineoplastic agent. Classical antineoplastic agents include but are not limited to hormonal modulators such as hormonal, anti-hormonal, androgen agonist, androgen antagonist and anti-estrogen therapeutic agents, histone deacetylase (HDAC) inhibitors, gene silencing agents or gene activating agents, ribonucleases, proteosomics, Topoisomerase I inhibitors, Camptothecin derivatives, Topoisomerase II inhibitors, alkylating agents, antimetabolites, poly(ADP-ribose) polymerase-1 (PARP-1) inhibitor, microtubulin inhibitors, antibiotics, plant derived spindle inhibitors, platinum-coordinated compounds, gene therapeutic agents, antisense oligonucleotides, vascular targeting agents (VTAs), and statins.

Examples of classical antineoplastic agents that may be used according to the methods disclosed herein include, but are not limited to, glucocorticoids, such as dexamethasone, prednisone, prednisolone, methylprednisolone, hydrocortisone, and progestins such as medroxyprogesterone, megestrol acetate (Megace), mifepristone (RU-486), Selective Estrogen Receptor Modulators (SERMs; such as tamoxifen, raloxifene, lasofoxifene, afimoxifene, arzoxifene, bazedoxifene, fispemifene, ormeloxifene, ospemifene, tesmilifene, toremifene, trilostane and CHF 4227 (Cheisi)), Selective Estrogen-Receptor Downregulators (SERD's; such as fulvestrant), exemestane (Aromasin), anastrozole (Arimidex), atamestane, fadrozole, letrozole (Femara), gonadotropin-releasing hormone (GnRH; also commonly referred to as luteinizing hormone-releasing hormone [LHRH]) agonists such as buserelin (Suprefact), goserelin (Zoladex), leuprorelin (Lupron), and triptorelin (Trelstar), abarelix (Plenaxis), bicalutamide (Casodex), cyproterone, flutamide (Eulexin), megestrol, nilutamide (Nilandron), and osaterone, dutasteride, epristeride, finasteride, Serenoa repens, PHL 00801, abarelix, goserelin, leuprorelin, triptorelin, bicalutamide, tamoxifen, exemestane, anastrozole, fadrozole, formestane, letrozole, and combinations thereof.

Other examples of classical antineoplastic agents that may be used according to the methods disclosed herein include, but are not limited to, suberolanilide hydroxamic acid (SAHA, Merck Inc./Aton Pharmaceuticals), depsipeptide (FR901228 or FK228), G2M-777, MS-275, pivaloyloxymethyl butyrate and PXD-101; Onconase (ranpirnase), PS-341 (MLN-341), Velcade (bortezomib), 9-aminocamptothecin, belotecan, BN-80915 (Roche), camptothecin, diflomotecan, edotecarin, exatecan (Daiichi), gimatecan, 10-hydroxycamptothecin, irinotecan HCl (Camptosar), lurtotecan, Orathecin (rubitecan, Supergen), SN-38, topotecan, camptothecin, 10-hydroxycamptothecin, 9-aminocamptothecin, irinotecan, SN-38, edotecarin, topotecan, aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide, AMD-473, altretamine, AP-5280, apaziquone, brostallicin, bendamustine, busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin, Eloxatin (oxaliplatin, Sanofi), streptozocin, satrplatin, and combinations thereof.

In some embodiments, the at least one additional medicinal or therapeutic agent includes dihydrofolate reductase inhibitors (such as methotrexate and NeuTrexin (trimetresate glucuronate)), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda®), cytosine arabinoside, Gemzar® (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, N-(5-[N-(3,4-dihydro-2-methyl-4-oxoquinazolin-6-ylmethyl)-N-methylamino]-2-thenoyl)-L-glutamic acid, AG-014699 (Pfizer Inc.), ABT-472 (Abbott Laboratories), INO-1001 (Inotek Pharmaceuticals), KU-0687 (KuDOS Pharmaceuticals) and GPI 18180 (Guilford Pharm Inc) and combinations thereof.

Other examples of classical antineoplastic cytotoxic agents used according to the methods disclosed herein include, but are not limited to, Abraxane (Abraxis BioScience, Inc.), Batabulin (Amgen), EPO 906 (Novartis), Vinflunine (Bristol-Myers Squibb Company), actinomycin D, bleomycin, mitomycin C, neocarzinostatin (Zinostatin), vinblastine, vincristine, vindesine, vinorelbine (Navelbine), docetaxel (Taxotere), Ortataxel, paclitaxel (including Taxoprexin a DHA/paciltaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta®), DMXAA (Antisoma), ibandronic acid, L-asparaginase, pegaspargase (Oncaspar®), Efaproxiral (Efaproxyn®—radiation therapy)), bexarotene (Targretin®), Tesmilifene (DPPE—enhances efficacy of cytotoxics)), Theratope® (Biomira), Tretinoin (Vesanoid®), tirapazamine (Trizaone®), motexafin gadolinium (Xcytrin®) Cotara® (mAb), and NBI-3001 (Protox Therapeutics), polyglutamate-paclitaxel (Xyotax®) and combinations thereof.

Further examples of classical antineoplastic agents that may be used according to the methods disclosed herein include, but are not limited to, as Advexin (ING 201), TNFerade (GeneVec, one or more compounds which express TNFalpha in response to radiotherapy), RB94 (Baylor College of Medicine), Genasense (Oblimersen, Genta), Combretastatin A4P (CA4P), Oxi-4503, AVE-8062, ZD-6126, TZT-1027, Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin, Niacin (Advicor, Kos Pharmaceuticals), Caduet, Lipitor, torcetrapib, and combinations thereof.

Some embodiments relate to a method for the treatment of breast cancer in a subject in need of such treatment, comprising administering to said subject an amount of one or more topical formulations disclosed herein in combination with one or more (preferably one to three) anti-cancer agents selected from the group consisting of trastuzumab, tamoxifen, docetaxel, paclitaxel, capecitabine, gemcitabine, vinorelbine, exemestane, letrozole and anastrozole.

Some embodiments provide a method of treating colorectal cancer in a subject in need of such treatment, by administering a topical formulation disclosed herein in combination with one or more (preferably one to three) anti-cancer agents. Examples of particular anti-cancer agents include those typically used in adjuvant chemotherapy, such as FOLFOX, a combination of 5-fluorouracil (5-FU) or capecitabine (Xeloda), leucovorin and oxaliplatin (Eloxatin). Further examples of particular anti-cancer agents include those typically used in chemotherapy for metastatic disease, such as FOLFOX or FOLFOX in combination with bevacizumab (Avastin); and FOLFIRI, a combination of 5-FU or capecitabine, leucovorin and irinotecan (Camptosar). Further examples include 17-DMAG, ABX-EFR, AMG-706, AMT-2003, ANX-510 (CoFactor), aplidine (plitidepsin, Aplidin), Aroplatin, axitinib (AG-13736), AZD-0530, AZD-2171, bacillus Calmette-Guerin (BCG), bevacizumab (Avastin), BIO-117, BIO-145, BMS-184476, BMS-275183, BMS-528664, bortezomib (Velcade), C-1311 (Symadex), cantuzumab mertansine, capecitabine (Xeloda), cetuximab (Erbitux), clofarabine (Clofarex), CMD-193, combretastatin, Cotara, CT-2106, CV-247, decitabine (Dacogen), E-7070, E-7820, edotecarin, EMD-273066, enzastaurin (LY-317615) epothilone B (EPO-906), erlotinib (Tarceva), flavopyridol, GCAN-101, gefitinib (Iressa), huA33, huC242-DM4, imatinib (Gleevec), indisulam, ING-1, irinotecan (CPT-11, Camptosar) ISIS 2503, ixabepilone, lapatinib (Tykerb), mapatumumab (HGS-ETR1), MBT-0206, MEDI-522 (Abregrin), Mitomycin, MK-0457 (VX-680), MLN-8054, NB-1011, NGR-TNF, NV-1020, oblimersen (Genasense, G3139), OncoVex, ONYX 015 (CI-1042), oxaliplatin (Eloxatin), panitumumab (ABX-EGF, Vectibix), pelitinib (EKB-569), pemetrexed (Alimta), PD-325901, PF-0337210, PF-2341066, RAD-001 (Everolimus), RAV-12, Resveratrol, Rexin-G, S-1 (TS-1), seliciclib, SN-38 liposome, Sodium stibogluconate (SSG), sorafenib (Nexavar), SU-14813, sunitinib (Sutent), temsirolimus (CCI 779), tetrathiomolybdate, thalomide, TLK-286 (Telcyta), topotecan (Hycamtin), trabectedin (Yondelis), vatalanib (PTK-787), vorinostat (SAHA, Zolinza), WX-UK1, and ZYC300, wherein the amounts of the active agent together with the amounts of the combination anticancer agents are effective in treating colorectal cancer.

Some embodiments provide methods for the treatment of renal cell carcinoma in a subject in need of such treatment, comprising administering to said subject a topical formulation disclosed herein in combination with one or more (preferably one to three) anti-cancer agents selected from the group consisting of capecitabine (Xeloda), interferon alpha, interleukin-2, bevacizumab (Avastin), gemcitabine (Gemzar), thalidomide, cetuximab (Erbitux), vatalanib (PTK-787), Sutent, AG-13736, SU-11248, Tarceva, Iressa, Lapatinib and Gleevec, wherein the amounts of the active agent together with the amounts of the combination anticancer agents is effective in treating renal cell carcinoma.

Some embodiments provide methods for the treatment of melanoma in a subject in need of such treatment, comprising administering to said subject a topical formulation as disclosed herein in combination with one or more (preferably one to three) anti-cancer agents selected from the group consisting of interferon alpha, interleukin-2, temozolomide (Temodar), docetaxel (Taxotere), paclitaxel, Dacarbazine (DTIC), carmustine (also known as BCNU), Cisplatin, vinblastine, tamoxifen, PD-325,901, Axitinib, bevacizumab (Avastin), thalidomide, sorafanib, vatalanib (PTK-787), Sutent, CpG-7909, AG-13736, Iressa, Lapatinib and Gleevec, wherein the amounts of the compound disclosed herein, or a pharmaceutically acceptable salt thereof, together with the amounts of the combination anticancer agents is effective in treating melanoma.

Some embodiments provide methods for the treatment of lung cancer in a subject in need of such treatment, comprising administering to said subject a topical formulation disclosed herein in combination with one or more (preferably one to three) anti-cancer agents selected from the group consisting of capecitabine (Xeloda), bevacizumab (Avastin), gemcitabine (Gemzar), docetaxel (Taxotere), paclitaxel, premetrexed disodium (Alimta), Tarceva, Iressa, Vinorelbine, Irinotecan, Etoposide, Vinblastine, and Paraplatin (carboplatin), wherein the amounts of the active agent together with the amounts of the combination anticancer agents is effective in treating lung cancer.

Some embodiments provide methods for the treatment of basal cell carcinoma in a subject in need of such treatment, comprising administering to said subject a topical formulation disclosed herein in combination with one or more additional medicinal or pharmaceutical agents selected from 5-fluorouracil, vismodegib, sonidegib, and imiquimod. In some embodiments, the one additional medicinal or pharmaceutical agent is 5-fluorouracil. In some embodiments, the one additional medicinal or pharmaceutical agent is vismodegib. In some embodiments, the one additional medicinal or pharmaceutical agent is sonidegib. In some embodiments, the one additional medicinal or pharmaceutical agent is imiquimod.

The examples herein are provided to illustrate advantages of the present technology and to further assist a person of ordinary skill in the art with using 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof. The examples herein is also presented in order to more fully illustrate the preferred aspects of the present technology. The example should in no way be construed as limiting the scope of the present technology, as defined by the appended claims. The example can include or incorporate any of the variations, aspects or aspects of the present technology described above. The variations, aspects or aspects described above may also further each include or incorporate the variations of any or all other variations, aspects or aspects of the present technology.

### EXAMPLES

### Example 1. Solubility Studies of 4-fluoro-N-methyl-N-(1-(4-(1-methyl-1H-pyrazol-5-yl)phthalazin-1-yl)piperidin-4-yl)-2-(trifluoromethyl)benzamide

The solubility of 4-fluoro-N-methyl-N-(1-(4-(1-methyl-1H-pyrazol-5-yl)phthalazin-1-yl)piperidin-4-yl)-2-(trifluoromethyl)benzamide was assessed in common topical excipients (Table 1) and in cosolvent mixtures, aqueous mixtures, and lipophilic mixtures (Table 2). The results from Table 2 suggest that solubility is highest in ethanol/water mixtures.

TABLE 1

| Solvents (v/v) | Solubility, mg/g |
|---|---|
| NMP | 107.02 |
| DMSO | 27.65 |
| EtOH | 13.66 |
| EtOH-Water (3:1) | 18.76 |
| EtOH-Water (1:1) | 2.51 |
| Isopropyl alcohol | 5.15 |
| IPA-Water (3:1) | 18.73 |

### Example 2. Chemical Stability of Alcohol-Based Formulations of 4-fluoro-N-methyl-N-(1-(4-(1-methyl-1H-pyrazol-5-yl)phthalazin-1-yl)piperidin-4-yl)-2-(trifluoromethyl)benzamide

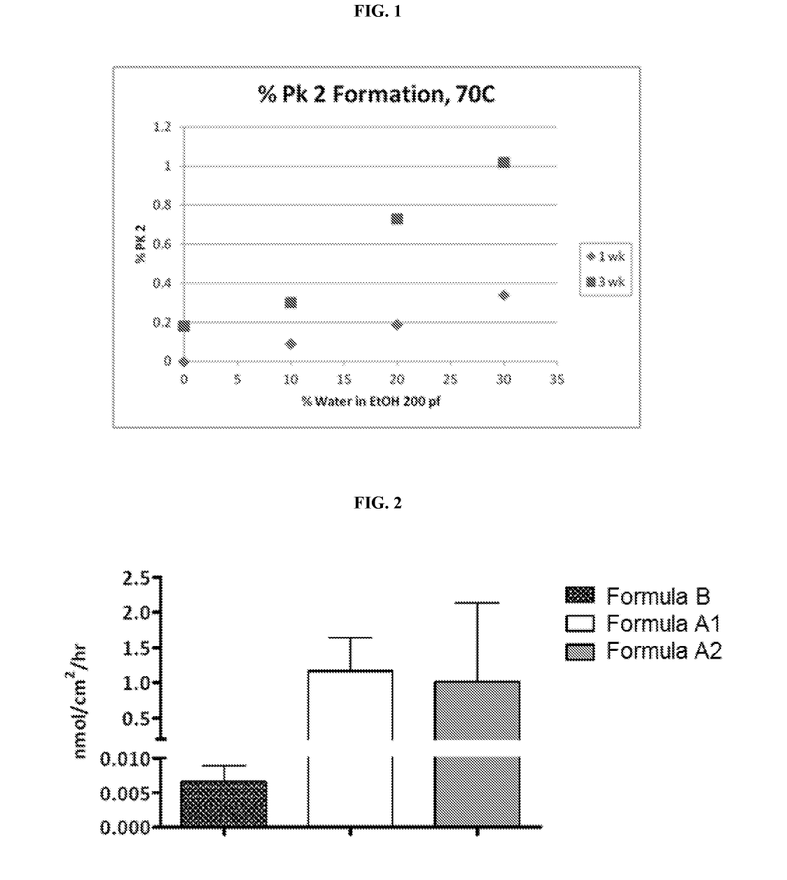

Various ethanol-based formulations were examined (Table 3). However, the extent of solvolysis of 4-fluoro-N-methyl-N-(1-(4-(1-methyl-1H-pyrazol-5-yl)phthalazin-1-yl)piperidin-4-yl)-2-(trifluoromethyl)benzamide increased with the percentage of water in formulations 1-4 following storage at 70° C. for 1 week or 3 weeks (FIG. 1). Formulation #5 demonstrated comparable results to formulation #1 after 1 week storage at 70° C.

TABLE 3

| | Formulations | | | | | | | |
|---|---|---|---|---|---|---|---|---|
| | 1 | 2 | 3 | 4 | 5 | 6 | 7 | 8 |
| API* | 0.5** | 0.5 | 0.5 | 0.5 | 0.5 | 0.5 | 0.5 | 0.5 |
| purified water | | 10.0 | 20.0 | 30.0 | 10.0 | 10.0 | | 10.0 |
| ethanol | | | | | 74.6 | 73.6 | | 20.0 |
| (190 proof) | | | | | | | | |
| ethanol | 97.5 | 87.5 | 77.5 | 67.5 | | | 87.5 | |
| (200 proof) | | | | | | | | |
| propylene | | | | | 12.9 | 12.9 | | 67.5 |
| glycol | | | | | | | | |
| glycerin | | | | | | | 10.0 | |
| benzyl alcohol | | | | | | 1.0 | | |
| HPC MF | 2.0 | 2.0 | 2.0 | 2.0 | 2.0 | 2.0 | 2.0 | 2.0 |
| Total | 100.0 | 100.0 | 100.0 | 100.0 | 100.0 | 100.0 | 100.0 | 100.0 |

*API = 4-fluoro-N-methyl-N-(1-(4-(1-methyl-1H-pyrazol-5-yl)phthalazin-1-yl)piperidin-4-yl)-
2-(trifluoromethyl)benzamide
**Amounts shown as % w/w

Stress testing with pure excipients was also conducted. Adducts were observed with excipients containing unhindered hydroxyl groups (e.g., ethanol, benzyl alcohol, Transcutol®). However, no adduct was observed with isopropyl alcohol.

### Example 4. Human Cadaver Skin Permeability Studies with 4-fluoro-N-methyl-N-(1-(4-(1-methyl-1H-pyrazol-5-yl)phthalazin-1-yl)piperidin-4-yl)-2-(trifluoromethyl)benzamide

Two formulations of 4-fluoro-N-methyl-N-(1-(4-(1-methyl-1H-pyrazol-5-yl)phthalazin-1-yl)piperidin-4-yl)-2-(trifluoromethyl)benzamide (Formulation A1: API (1.8% w/w) in IPA/water/isopropylmyristate (IPM)/HPC (73.2:20:3:2); Formulation A2: API (1.8% w/w) in IPA/water/isopropylmyristate (IPM)/propylene carbonate (PC)/HPC (71.2:15:3:7:2)) and a cream formulation of LDE225 (Formulation B; as described in WO2011009852) were compared for permeability and tissue concentrations in human skin. Static, vertical glass Franz diffusion cells were used for the permeability assay. Dermatomed skin from a post-mortem human donor (Caucasian male, age 52, cause of death: end-stage liver disease, ethanol-induced cirrhosis of liver) was used for this study.

The dosing solution with control compounds (atenolol and testosterone, 100 μM each) was prepared in PBS buffer at pH 7.4 immediately prior to the start of the permeability experiments. The treatment groups are shown in Table 4. The skin was kept frozen at −80° C. until the time of the study. The skin was thawed at room temperature and cut to appropriate size (˜2×2 cm). The thickness of each piece was measured and they were grouped based on thickness to ensure that the average thickness was similar across groups. Tissue was mounted in the Franz cell diffusion chamber and clamped between the donor and receiver chambers. The exposed surface area of the Franz cell diffusion chamber was 1.77 cm². The receiver compartment was filled with 8 mL PBS at pH 7.4 with 1% Oleth 20 for all test groups, or PBS at pH 7.4 for the control group. The reservoir also contained a magnetic stir bar, stirring at 400 RPM to ensure homogeneity of the reservoir contents. Each Franz cell diffusion chamber was placed in a dry block heating/stirring module with temperature set to maintain the tissue surface at approximately 32° C.

Each formulated test compound (35.4 μL by volume) was dispensed into the donor chamber using the reverse pipetting technique, after warming the cream in a water bath at 37° C. The control solution (2 mL) was added to the donor chamber of the control group. All chambers were covered to minimize evaporation. Samples (1 mL) were taken from the receiver compartment at 2, 4, 8, 24, and 30 hours. After each sampling, an equal volume of fresh buffer was added to maintain sink conditions. The donor compartment of the control group was sampled at the onset and end of the assay. Each formulation was also analyzed to verify the concentration of the test compound. At the end of the permeability assay, skin treated with formulated test compound was removed from the chamber, wiped of excess test material, and rinsed in saline. Each piece of skin treated with test formulation was tape-stripped twice to remove residual test compound that was not washed away by the saline rinse. Each specimen was then immersed individually in distilled water at 61±1° C. for 1 to 2 min, blotted dry, and separated into dermis and epidermis using a pair of fine forceps. Each skin layer was weighed and stored at −80° C. until analysis.

TABLE 4

| Chamber # | Group | Donor | Receiver |
|---|---|---|---|
| 1 | Formulation B | 20 μL/cm² | 8 mL of PBS, |
| 2 | (LDE225) | (35.4 μL/chamber) | pH 7.4 + |
| 3 | | | 1% Oleth 20 |
| 4 | | | |
| 5 | Formulation A1 | | |
| 6 | | | |
| 7 | | | |
| 8 | | | |
| 9 | Formulation A2 | | |
| 10 | | | |
| 11 | | | |
| 12 | | | |
| 21 | Controls | 2 mL in PBS, | 8 mL of PBS, |
| 22 | (atenolol and | pH 7.4 | pH 7.4 |
| 23 | testosterone, | | |
| 24 | 100 μM each) | | |

The flux values for each formulated compound are shown in Table 5 and FIG. 2. Formulation B (LDE225) exhibited significantly lower flux than Formulation A1 (P<0.01). The flux of LDE225 tended to be lower than that of Formulation A2. Formulation A1 had a similar flux to that of Formulation A2, although the latter was associated with higher variability among replicates.

TABLE 5

| | Replicate | | | | |
|---|---|---|---|---|---|
| Treatment Group | 1 | 2 | 3 | 4 | mean ± SD |
| Formulation B | 0.0039 | 0.0070 | 0.0059 | 0.0094 | 0.0065 ± 0.0023 |
| Formulation A1 | 0.66 | 1.4 | 0.91 | 1.7 | 1.2 ± 0.47 |
| Formulation A2 | 0.23 | 0.23 | 1.0 | 2.6* | 1.0 ± 1.1 |

*This number was tested by Q-test for outliers, but it was not a significant outlier.

The Pₐₚₚ and recovery values of the control compounds indicated that the integrity of the skin tissue was maintained. Pₐₚₚ of atenolol for each replicate was less than 0.2×10⁻⁶ cm/sec and the ratio of testosterone to atenolol Pₐₚₚ was greater than 2.

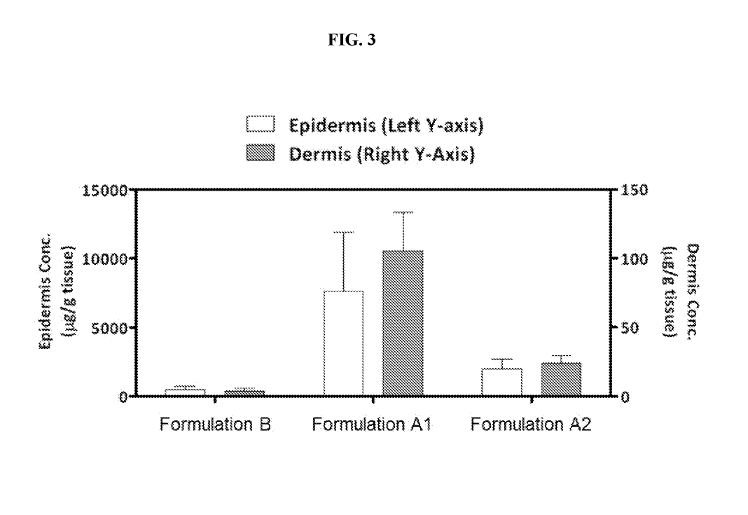

The accumulation of the two test compounds in the epidermis and dermis layers is presented in Table 6 and FIG. 3. The concentration of the test compounds is expressed as the amount (μg) normalized by the tissue weight of each layer (g).

TABLE 6

| | | Epidermis | | | Dermis | | |
|---|---|---|---|---|---|---|---|
| | | Tissue | | | Tissue | | |
| | | Conc. | | | Conc. | | |
| Treatment | Repli- | (μg/g | | | (μg/g | | |
| Group | cate | tissue) | Mean | SD | tissue) | Mean | SD |
| Form- | 1 | 390 | 459 | 246 | 1.0 | 3.7 | 1.9 |
| ulation B | 2 | 791 | | | 3.7 | | |
| | 3 | 453 | | | 4.5 | | |
| | 4 | 201 | | | 5.4 | | |
| Form- | 1 | 7534 | 7633 | 4282 | 90.9 | 105.3 | 28.2 |
| ulation A1 | 2 | 4085 | | | 146.6 | | |
| | 3 | 5228 | | | 99.4 | | |
| | 4 | 13684 | | | 84.2 | | |
| Form- | 1 | 1135 | 2009 | 682 | 16.9 | 23.7 | 5.9 |
| ulation A2 | 2 | 2539 | | | 27.2 | | |
| | 3 | 1799 | | | 27.0 | | |
| | 4 | 2563 | | | 130.2* | | |

*Data point was identified as an outlier (Q-test) and was not included in the calculation of mean or SD

The accumulation of LDE225 in epidermis was significantly less than that of 4-fluoro-N-methyl-N-(1-(4-(1-methyl-1H-pyrazol-5-yl)phthalazin-1-yl)piperidin-4-yl)-2-(trifluoromethyl)benzamide, either from Formulation A1 (P<0.05) or from Formulation A2 (P<0.01). Between Formulations A1 and A2, significantly more 4-fluoro-N-methyl-N-(1-(4-(1-methyl-1H-pyrazol-5-yl)phthalazin-1-yl)piperidin-4-yl)-2-(trifluoromethyl)benzamide accumulated in epidermis from Formulation A1 than from Formulation A2 (P<0.05).

In the dermis, significantly less LDE225 accumulated than 4-fluoro-N-methyl-N-(1-(4-(1-methyl-1H-pyrazol-5-yl)phthalazin-1-yl)piperidin-4-yl)-2-(trifluoromethyl)benzamide, either from Formulation A1 (P<0.001) or from Formulation A2 (P<0.01). Between Formulations A1 and A2, significantly more 4-fluoro-N-methyl-N-(1-(4-(1-methyl-1H-pyrazol-5-yl)phthalazin-1-yl)piperidin-4-yl)-2-(trifluoromethyl)benzamide accumulated in dermis from Formulation A1 than from Formulation A2 (P<0.01).

### Example 5. PK/PD Response in Gottingen Minipigs Following 7-Day Repeat Topical Dosing of 4-fluoro-N-methyl-N-(1-(4-(1-methyl-1H-pyrazol-5-yl)phthalazin-1-yl)piperidin-4-yl)-2-(trifluoromethyl)benzamide Formulated in Different Topical Excipients and Compared to LDE225

This study was conducted to compare the skin biomarker (Gli-1) effects following intradermal or topical delivery of compound. Thirteen formulations shown in Table 7 were assessed in this study.

TABLE 7

| | | Concentration |
|---|---|---|
| Formulation | Composition (% w/w)** | (15 mg/mL)* |
| 1 | IPA/water/IPM/HPC | 1.8% API^{†} |
| | (75.2/18.0/3.0/2.0) | |
| 2 | IPA/water/IPM/HPC (occluded) | 1.8% API |
| | (75.2/18.0/3.0/2.0) | |
| 3 | IPA/water/IPM/HPC (micro-needle) | 1.8% API |
| | (75.2/18.0/3.0/2.0) | |
| 4 | EtOH/water/IPM/HPC | 1.8% API |
| | (75.2/18.0/3.0/2.0) | |
| 5 | IPA/water/BA/IPM/HPC | 1.8% API |
| | (72.2/18.0/3.0/3.0/2.0) | |
| 6 | IPA/water/IPM/MYRJ ™ 52/HPC | 1.8% API |
| | (74.7/18.0/3.0/0.5/2.0) | |
| 7 | IPA/water/PC/IPM/HPC | 1.8% API |
| | (68.2/18.0/7.0/3.0/2.0) | |
| 8 | IPA/water/PC/HPC | 1.8% API |
| | (66.0/23.2/7.0/2.0) | |
| 9 | IPA/water/DMSO/HPC | 0.9% API* |
| | (59.1/18/20.0/2.0) | (7.5 mg/mL) |
| 10 | IPA/water/Octisalate/HPC | 1.8% API |
| | (72.2/18.0/6/2.0) | |
| 11 | IPA/water/BA/HPC | 7.2% API* |
| | (43/30/25/2) | (60 mg/mL) |
| 12 | 0.5% Pluronic ® F68 in | 200 mg/mL API* |
| | saline (intradermal) | |
| 13 | LDE225 cream | 0.75% LDE225* |

**Abbreviations: IPA = isopropyl alcohol; IPM = isopropylmyristate; HPC = hydroxypropyl cellulose; EtOH = ethanol (190 proof); BA = benzyl alcohol; PC = propylene carbonate; DMSO = dimethyl sulfoxide
*Concentration is 15 mg/mL unless otherwise noted.
^{†}API = 4-fluoro-N-methyl-N-(1-(4-(1-methyl-1H-pyrazol-5-yl)phthalazin-1-yl)piperidin-4-yl)-2-(trifluoromethyl)benzamide

Eight female and eight male non-naïve Gottingen Minipigs (at least 4 months of age) were used for this study. Animals were not fasted prior to dosing. Formulations 1-11 and 13 from Table 7 were administered topically. Formulation 12 was administered intradermally. Formulations 2 and 3 were administered once daily (QD) on days 2 through 8. Formulation 12 was administered as a single dose on day 1. Formulations 1, 4-11, and 13 were administered once on days 1 and 8, and twice daily (BID) (approximately 8 hours apart) on days 2 through 7.

Topical administration: on the day prior to dose administration, the hair was clipped from the back of all animals. All animals were shaved again on the day prior to the end of study biopsies. The exposure sites were marked at the corners with an indelible marker. The dosing formulations were distributed over the prescribed area (approximately 4 cm by 4 cm) by gentle inunction with a glass stirring rod. The treatment area was not occluded and at approximately 24 hours following the first dose and every 24 hours thereafter, the dosing site was washed. Formulations 1, 4-11 and 13 were administered to the appropriate animals at 0.4 mL per animal BID.

Topical administration (occluded): following dose administration of formulation 2, the site was allowed to dry for 2 minutes and a waterproof bandage was applied to cover the entire dosing area. The bandage was removed prior to the next dermal wash and dose. Formulation 2 was administered to the appropriate animals at 0.4 mL per animal QD.

Topical administration (micro-needle): prior to administration of formulation 3, the application site was prepared using the Derma Roller Micro Needle Roller System, Model OR 50. After the site was cleaned with IPA, the tool was pressed firmly and rolled over the application site vertically and horizontally three times to ensure the entire site was prepared. A new tool was used daily and the tool was cleaned with chlorhexidine scrub and rinsed with sterile saline between each animal. Formulation 3 was administered to the appropriate animals at 0.4 mL per animal QD.

Intradermal (ID) administration: prior to dosing, anesthesia was induced using telazol at 3 to 5 mg/kg for all animals on day 1 to aid with dose administration. The ID exposure site was marked at the site of injection with an indelible marker for aid in biopsy sampling. Formulation 12 was administered to the appropriate animals at 0.15 mL, 0.2 mL, 0.1 mL, or 0.07 mL once on day 1.

Punch biopsy collection from all animals was conducted pretest and on day 8, approximately 8 hours post the last dose. Blood samples were collected from all animals on day 7, approximately 8 hours post the last dose.

Formulations 4, 7, and 9 were further investigated.

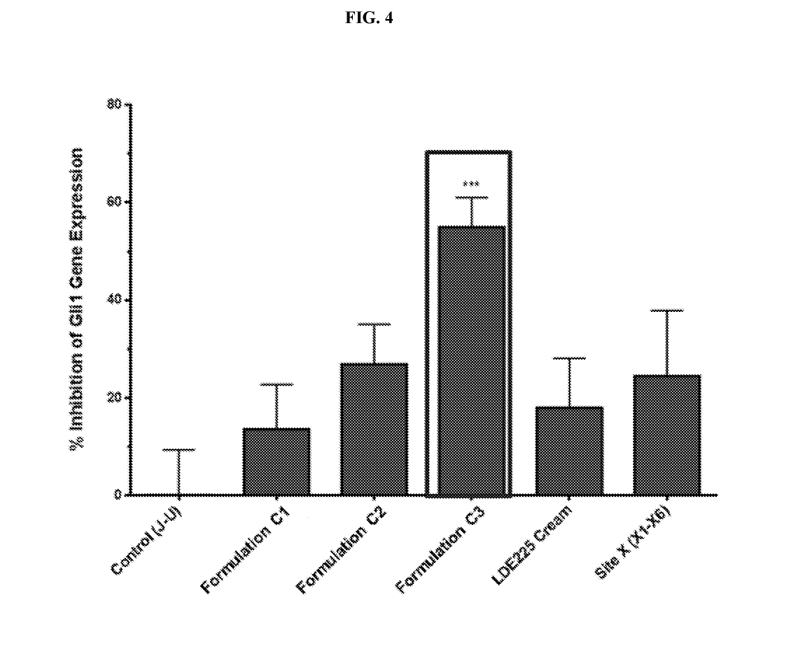

Three formulations and a control cream formulation for LDE225 (Table 8) underwent a second minipig study. Analysis of tissue samples (select data in FIG. 4) indicated that formulation C3 (containing DMSO) exhibited the highest percent inhibition of Gli1 gene expression and was superior to that of the LDE225 cream formulation.

TABLE 8

| | Composition (% w/w)** | Concentration |
|---|---|---|
| Formulation C1 | IPA/water/IPM/HPC | 1.8% API^{†} (15 mg/mL) |
| | (75.2/18.0/3.0/2.0) | |
| Formulation C2 | IPA/water/IPM/PC/HPC | 1.8% API (15 mg/mL) |
| | (70.2/18.0/3.0/5.0/2.0) | |
| Formulation C3 | IPA/water/DMSO/HPC | 0.9% API (7.5 mg/mL) |
| | (59.1/18.0/20.0/2.0) | |
| Formulation D | “Cream B”* | 0.75% LDE225 |

**Abbreviations: IPA = isopropyl alcohol; IPM = isopropylmyristate; HPC = hydroxypropyl cellulose; PC = propylene carbonate; DMSO = dimethyl sulfoxide
*Disclosed in WO2011009852
^{†}API = 4-fluoro-N-methyl-N-(1-(4-(1-methyl-1H-pyrazol-5-yl)phthalazin-1-yl)piperidin-4-yl)-2-(trifluoromethyl)benzamide

### Example 6. Further Investigation of IPA/DMSO Formulation

Investigations indicated that at concentrations above 20% DMSO and with increasing concentration of DMSO, there was increased 4-fluoro-N-methyl-N-(1-(4-(1-methyl-1H-pyrazol-5-yl)phthalazin-1-yl)piperidin-4-yl)-2-(trifluoromethyl)benzamide solubility in IPA/DMSO solutions in the absence of water. However, as the percentage of water increased (5%, 10%, 20% or 30% water), the IPA/water/DMSO solutions demonstrated less solubility of 4-fluoro-N-methyl-N-(1-(4-(1-methyl-1H-pyrazol-5-yl)phthalazin-1-yl)piperidin-4-yl)-2-(trifluoromethyl)benzamide.

IPA/DMSO formulations were stable for 6 months at 40° C., 50° C., and 70° C., with a predicted shelf life of at least three years. Freeze-thaw of IPA/DMSO formulations showed no precipitation, even after 6 months at 5° C. or −70° C.

A minipig study was performed using three IPA/DMSO formulations of 4-fluoro-N-methyl-N-(1-(4-(1-methyl-1H-pyrazol-5-yl)phthalazin-1-yl)piperidin-4-yl)-2-(trifluoromethyl)benzamide and two placebo formulations with no compound (Table 9). No adverse findings were observed with any of these formulations.

TABLE 9

| | Placebo | Placebo | Formulation | Formulation | Formulation |
|---|---|---|---|---|---|
| Ingredient | 1 | 2 | E1 | E2 | E3 |
| API* | 0.00** | 0.00 | 0.45 | 0.90 | 1.80 |
| purified | 18.00 | 0.00 | 18.00 | 18.00 | 0.00 |
| water | | | | | |
| IPA, USP | 60.00 | 48.00 | 59.55 | 59.10 | 46.20 |
| DMSO, | 20.00 | 50.00 | 20.00 | 20.00 | 50.00 |
| USP | | | | | |
| HPC | 2.00 | 2.00 | 2.00 | 2.00 | 2.00 |
| TOTAL | 100.00 | 100.00 | 100.00 | 100.00 | 100.00 |

*API = 4-fluoro-N-methyl-N-(1-(4-(1-methyl-1H-pyrazol-5-yl)phthalazin-1-yl(piperidin-4-yl)-2-(trifluoromethyl)benzamide
**Amounts shown as % w/w

### Example 7. Topical Gel Formulation Comprising 0.45% w/w of 4-fluoro-N-methyl-N-(1-(4-(1-methyl-1H-pyrazol-5-yl)phthalazin-1-yl)piperidin-4-yl)-2-(trifluoromethyl)benzamide

The following ingredients were combined in a stainless steel mixing vessel: dimethyl sulfoxide, USP; isopropyl alcohol, 99%, USP; and 4-fluoro-N-methyl-N-(1-(4-(1-methyl-1H-pyrazol-5-yl)phthalazin-1-yl)piperidin-4-yl)-2-(trifluoromethyl)benzamide. The ingredients were mixed using an overhead mixer. The vessel was covered as much as possible to avoid evaporation and to protect the gel from light. While stirring, hydroxypropyl cellulose (HPC) (Klucel™) was slowly added to the mixture. The gel was stirred until the hydroxypropyl cellulose was evenly dispersed into small agglomerates. The gel was then further mixed to disrupt these small agglomerates until an even dispersion was visually confirmed. The gel was allowed to sit for at least 15 min to allow the gel to thicken and air bubbles to dissipate. It was acceptable to have air bubbles dispersed throughout the gel. In the absence of air bubbles, the gel was clear and transparent. The final composition of this gel formulation was 0.45% w/w of 4-fluoro-N-methyl-N-(1-(4-(1-methyl-1H-pyrazol-5-yl)phthalazin-1-yl)piperidin-4-yl)-2-(trifluoromethyl)benzamide; 47.55% w/w of isopropyl alcohol, 99%, USP; 50.0% w/w of dimethyl sulfoxide, USP; and 2.0% w/w of hydroxypropyl cellulose (Klucel™).

### Example 8. Topical Gel Formulation Comprising 0.9% w/w of 4-fluoro-N-methyl-N-(1-(4-(1-methyl-1H-pyrazol-5-yl)phthalazin-1-yl)piperidin-4-yl)-2-(trifluoromethyl)benzamide

The following ingredients were combined in a stainless steel mixing vessel: dimethyl sulfoxide, USP; isopropyl alcohol, 99%, USP; and 4-fluoro-N-methyl-N-(1-(4-(1-methyl-1H-pyrazol-5-yl)phthalazin-1-yl)piperidin-4-yl)-2-(trifluoromethyl)benzamide. The ingredients were mixed using an overhead mixer and heated slowly to no more than 50° C. Heating was discontinued once components were mixed. The vessel was covered as much as possible to avoid evaporation and to protect the gel from light. While stirring, hydroxypropyl cellulose (Klucel™) was slowly added to the mixture. The gel was stirred until the hydroxypropyl cellulose was evenly dispersed into small agglomerates. The gel was then further mixed to disrupt these small agglomerates until an even dispersion was visually confirmed. The gel was allowed to sit for at least 15 min to allow the gel to thicken and air bubbles to dissipate. It was acceptable to have air bubbles dispersed throughout the gel. In the absence of air bubbles, the gel was clear and transparent. The final composition of this gel formulation was 0.9% w/w of 4-fluoro-N-methyl-N-(1-(4-(1-methyl-1H-pyrazol-5-yl)phthalazin-1-yl)piperidin-4-yl)-2-(trifluoromethyl)benzamide; 47.10% w/w of isopropyl alcohol, 99%, USP; 50.0% w/w of dimethyl sulfoxide, USP; and 2.0% w/w of hydroxypropyl cellulose (Klucel™).

### Example 9. Topical Gel Formulation Comprising 1.8% w/w of 4-Fluoro-N-methyl-N-(1-(4-(1-methyl-1H-pyrazol-5-yl)phthalazin-1-yl)piperidin-4-yl)-2-(trifluoromethyl)benzamide

The following ingredients were combined in a stainless steel mixing vessel: dimethyl sulfoxide, USP; isopropyl alcohol, 99%, USP; and 4-fluoro-N-methyl-N-(1-(4-(1-methyl-1H-pyrazol-5-yl)phthalazin-1-yl)piperidin-4-yl)-2-(trifluoromethyl)benzamide. The ingredients were mixed using an overhead mixer and heated slowly to no more than 50° C. Heating was discontinued once components were mixed. The vessel was covered as much as possible to avoid evaporation and to protect the gel from light. While stirring, hydroxypropyl cellulose (Klucel™) was slowly added to the mixture. The gel was stirred until the hydroxypropyl cellulose was evenly dispersed into small agglomerates. The gel was then further mixed to disrupt these small agglomerates until an even dispersion was visually confirmed. The gel was allowed to sit for at least 15 min to allow the gel to thicken and air bubbles to dissipate. It was acceptable to have air bubbles dispersed throughout the gel. In the absence of air bubbles, the gel was clear and transparent. The final composition of this gel formulation was 1.8% w/w of 4-fluoro-N-methyl-N-(1-(4-(1-methyl-1H-pyrazol-5-yl)phthalazin-1-yl)piperidin-4-yl)-2-(trifluoromethyl)benzamide; 46.20% w/w of isopropyl alcohol, 99%, USP; 50.0% w/w of dimethyl sulfoxide, USP; and 2.0% w/w of hydroxypropyl cellulose (Klucel™).

### Example 10. Human Clinical Trial for Treatment of Basal Cell Carcinoma

A topical formulation disclosed herein is administered to patients with basal cell carcinoma. Down-regulation in Gli expression over 8-days is assessed as a primary outcome. Secondary outcome measures include incidence, timing, and severity of treatment adverse events over a time frame of 45 days.

Patient Inclusion Criteria
Participants must be over the age of 18 years.
Male.
Women who do not have child-bearing potential (history of hysterectomy, post-menopausal).
Have a biopsy confirmed BCC that measures at least 6 mm in size at the time of the initial evaluation (visit #1).
Participant must be willing and comply with the requirements of the protocol.
Participant must have the ability to understand and communicate with the investigator.
Participant must provide informed consent.

Patient Exclusion Criteria
Subject with significant congestive heart failure (CHF) or history of CHF, chronic renal failure, hepatic failure, neuropathy.
Subject with current skin diseases that the investigator feels is not safe for study participation including but not limited to severe atopic dermatitis, cutaneous T-cell lymphoma, erythroderma.
Subjects on systemic medications known to affect the Hedgehog pathway.
Subjects on cisapride, oral midazolam, nisoldipine, felodipine, pimozide, quinidine, dofetilide, triazolam, methadone, levacetylmethadol (levomethadyl), lovastatin, simvastatin, dihydroergotamine, ergometrine (ergonovine), ergotamine and methylergometrine (methylergonovine), cisapride, pimozide, methadone, levacetylmethadol (levomethadyl), quinidine.
Subjects with history of hypersensitivity to azoles.
Subjects with Gorlin syndrome.
Subjects on chronic immunosuppression, or who have a history of compromised immune function (e.g., history of or current malignancy other than BCC/squamous cell skin cancers).
Subjects who do not speak English or have difficulty hearing or are otherwise impaired for providing informed consent and communicating with the investigator.
Subjects with a history of keloids or excessive scarring.
Subjects with known allergy to lidocaine, epinephrine, itraconazole or petrolatum.
Women of child-bearing age/potential and/or able to conceive.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (c), etc., are presented merely for ease of reading the specification and claims. The use of headings in the specification or claims does not require the steps or elements be performed in alphabetical or numerical order or the order in which they are presented.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The pharmaceutical composition may include an effective amount of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof. In any of the above embodiments, the effective amount may be determined in relation to a subject.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are set forth in the following claims, along with the full scope of equivalents to which such claims are entitled.

1. A topical formulation comprising 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof; one or more diluents; and one or more gelling agents.

2. The topical formulation of claim 1, wherein 4-fluoro-N-methyl-N-(1-(4-(1-methyl-1H-pyrazol-5-yl)phthalazin-1-yl)piperidin-4-yl)-2-(trifluoromethyl)benzamide is present in an amount of from about 0.1% w/w to about 5% w/w.

3. (canceled)

4. The topical formulation of claim 1, wherein the one or more diluents comprise isopropyl alcohol, dimethyl sulfoxide, or a combination thereof.

5. The topical formulation of claim 1, wherein the one or more diluents are present in an amount of from about 40% w/w to about 99% w/w.

6. The topical formulation of claim 1, wherein the one or more gelling agents comprise hydroxypropyl cellulose.

7. The topical formulation of claim 1, wherein the one or more gelling agents are present in an amount of from about 0.5% w/w to about 10% w/w.

8. (canceled)

9. A pharmaceutical formulation comprising 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, and one or more gelling agents, wherein the pharmaceutical formulation is suitable for topical use in a subject.

10. The pharmaceutical formulation of claim 9, wherein 4-fluoro-N-methyl-N-(1-(4-(1-methyl-1H-pyrazol-5-yl)phthalazin-1-yl)piperidin-4-yl)-2-(trifluoromethyl)benzamide is present in an amount of from about 0.1% w/w to about 5% w/w.

11. (canceled)

12. The pharmaceutical formulation of claim 9, wherein the one or more gelling agents comprise hydroxypropyl cellulose.

13. The pharmaceutical formulation of claim 9, wherein the one or more gelling agents are present in an amount of from about 0.5% w/w to about 10% w/w.

14. The pharmaceutical formulation of claim 9, wherein the one or more gelling agents are present in an amount of from about 1% w/w to about 3% w/w.

15. The pharmaceutical formulation of claim 9 further comprising one or more diluents.

16. The pharmaceutical formulation of claim 15, wherein the one or more diluents comprise isopropyl alcohol, dimethyl sulfoxide, or a combination thereof.

17. The pharmaceutical formulation of claim 15, wherein the one or more diluents are present in an amount of from about 40% w/w to about 99% w/w.

18. A method of treating cancer in a subject, comprising topically administering to the subject a composition comprising a therapeutically effective amount of 4-fluoro-N-methyl-N-(1-(4-(1-methyl-1H-pyrazol-5-yl)phthalazin-1-yl)piperidin-4-yl)-2-(trifluoromethyl)benzamide, or a pharmaceutically acceptable salt thereof, and one or more gelling agents.

19. The method of claim 18, wherein the cancer is basal cell carcinoma.

20. The method of claim 19, wherein the basal cell carcinoma is selected from nodular, superficial, morpheaform, pigmented basal cell carcinoma, and Fibroepithelioma of Pinkus (FEP).

21. The method of claim 18, wherein the composition is administered to the subject once per day.

22. The method of claim 18, wherein the composition is administered to the subject once per day for 2 consecutive days.

23. The method of claim 18, wherein the composition is administered to the subject following surgery to treat the cancer.
